(19) United States
(12) Patent Application Publication
(10) Pub. No.: US 2025/0228759 A1
(43) Pub. Date: Jul. 17, 2025

(54) ANTI-AGING COSMETIC COMPOSITIONS COMPRISING XANTHOMMATIN

(71) Applicant: SEASPIRE, INC., Cambridge, MA (US)

(72) Inventors: Leila F. Deravi, Cambridge, MA (US); Camille A. Martin, Cambridge, MA (US); Michael Burns, Cambridge, MA (US); Natalie Cox, Cambridge, MA (US)

(21) Appl. No.: 18/291,393

(22) PCT Filed: Jul. 26, 2022
(86) PCT No.: PCT/US2022/038316

§ 371 Date: Jan. 23, 2024

Related U.S. Application Data
(60) Provisional application No. 63/226,061, filed on Jul. 27, 2021.

(51) Int. Cl. A61K 8/49; A61K 8/11; A61K 8/14; A61K 8/34; A61K 8/55; A61Q 19/08

(52) U.S. Cl. CPC A61K 8/49; A61K 8/11; A61K 8/14; A61K 8/345; A61K 8/553; A61Q 19/08; A61K 2800/21; A61K 2800/70; A61K 2800/805

(57) ABSTRACT

Provided herein are ingredient blends including a phenoxazone and/or phenoxazine compound, or a salt thereof, such as xanthommatin, or a salt thereof, for use in anti-aging products wherein, upon combination with one or more further cosmetic composition or topical dermatologic drug composition, the ingredient blend provides an effect on one or more sign of skin-aging. Also provided herein are methods of preparing anti-aging products using the ingredient blends.

### FUNDING

This invention was made with government support under Grant NSF SBIR 2050284 awarded by the National Science Foundation. The government has certain rights in the invention.

### RELATED APPLICATIONS

This application claims the benefit of priority to U.S. Provisional Patent Application No. 63/226,061, filed Jul. 27, 2021, which application is hereby incorporated by reference in its entirety.

### BACKGROUND

Intrinsic aging is the natural and chronological physiological decay process that takes place in our body over time. Skin undergoes many changes in morphology and physiology with intrinsic aging. As in other organs, skin aging can be accelerated by exposure to environmental factors such as UV radiation, air pollution, dry environment, etc. Skin aging accelerated by environmental factors, called premature or extrinsic aging, can be distinguished from chronological or intrinsic aging by the characteristic signs of each type of aging. Specifically, the signs of extrinsic aging are more pronounced on the skin and include thicker and rougher skin, formation of dark spots and stains, telangiectasia (presence of tiny dilated blood vessels on the skin), sagging and wrinkling of the skin.

Currently, retinoids and vitamin C are the most representative anti-aging functional raw materials for the treatment of wrinkles, building collagen, and treating hyperpigmentation. Among the retinoids, retinoic acid is used as a powerful anti-aging therapeutic agent. Retinoic acid, however, is limited as a therapeutic agent due to its strongly irritant nature. Retinol, retinyl palmitate, and similar derivatives have relatively less skin irritation and are widely used as cosmetic materials. When exposed to heat and/or light, however, retinol and retinyl palmitate are denatured and their efficacy diminishes. Additionally, they may be phototoxic, thereby causing irritation and damage to the skin. Similarly, while vitamin C is a widely used therapeutic agent for reducing wrinkles, it also causes skin irritation due to its strong acidity and has stability issues as an ingredient.

Accordingly, there remains a need for additional anti-aging cosmetic compositions that are safe and highly effective.

### SUMMARY

The present application provides an ingredient blend for use in an anti-aging product. The ingredient blend is formulated so as to be mixed, for example in a manufacturing facility, with one or more compositions to produce a finished cosmetic. In implementations, the ingredient blend includes an unsaturated solution of a phenoxazone and/or phenoxazine compound, or a salt thereof. Upon combination of the ingredient blend with one or more further cosmetic composition or topical dermatologic drug composition, the finished cosmetic (solely or largely due to the contribution of the ingredient blend) provides an effect on one or more signs of skin-aging.

In certain embodiments of the ingredient blends of the present application, the phenoxazone and/or phenoxazine compound, or a salt thereof, is encapsulated in a liposome. In certain such embodiments, the liposome is embedded within the at least one cross-linkable polymer. In certain embodiments of the present application wherein the phenoxazone and/or phenoxazine compound, or a salt thereof, is encapsulated in a liposome, the liposome comprises a phospholipid and a polyol. In certain embodiments, the phospholipid is selected from natural phospholipids and synthetic phospholipids. In certain embodiments, the phospholipid comprises one or more of egg yolk lecithin, soybean lecithin, sphingomyelin, hydrogenated or non-hydrogenated phosphatidylcholine, phosphatidylethanolamine, phosphatidylserine, phosphatidylglycerol, phosphatidylinositol, dimyristoylphosphatidylcholine (DMPC), distearoylphosphatidylcholine, palmitoyl-stearoylphosphatidylcholine, dipalmitoyl phosphatidylcholine, and hydrogenated lecithin. In certain embodiments, the phospholipid comprises lecithin. In certain such embodiments, the lecithin is a naturally derived unsaturated or saturated lecithin extracted from soybean or egg yolk. In certain embodiments, the polyol comprises one or more of propanediol, propylene glycol, dipropylene glycol, 1,3-butylene glycol, glycerin, methylpropanediol, isoprene glycol, pentylene glycol, erythritol, xylitol and sorbitol. In certain embodiments, the polyol comprises propanediol. In certain embodiments, the liposomes are less than 1000 nm in diameter. In certain embodiments, the liposomes have a mean diameter of 250 nm. In certain embodiments, the liposomes are from about 200 to about 400 nm in diameter. In certain embodiments, the liposomes have a pH of about 5 to about 8.

In certain embodiments, ingredient blends provide a phenoxazone and/or phenoxazine compound, or a salt thereof, selected from xanthommatin, decarboxylated xanthommatin, uncyclized xanthommatin, ommatin D, dihydroxy-xanthommatin, rhodommatin, a derivative of any of the foregoing, and a precursor of any of the foregoing, and salts of any of the foregoing. In certain such embodiments, the unsaturated solution of a phenoxazone and/or phenoxazine compound, or a salt thereof, comprises unaggregated molecules.

In certain embodiments, the phenoxazone and/or phenoxazine compound, or a salt thereof, is xanthommatin, or a salt thereof. In certain such embodiments, the phenoxazone and/or phenoxazine compound, or a salt thereof, is ammonium xanthommatin.

In certain embodiments, ingredient blends are water-based.

In certain embodiments, the phenoxazone and/or phenoxazine compound, or a salt thereof, comprises 0.01-1 wt % of the ingredient blend. In certain such embodiments, the phenoxazone and/or phenoxazine compound, or a salt thereof, compound comprises 0.4 wt % of the ingredient blend.

The present application further provides an ingredient blend for use in an anti-aging product comprising a water-based solution of xanthommatin, or a salt thereof, wherein, upon combination with one or more further cosmetic composition or topical dermatologic drug composition to provide a finished cosmetic. The finished cosmetic (through the contribution of the ingredient blend) provides an effect on one or more sign of skin-aging, and where the xanthommatin, or a salt thereof, comprises up to 1 wt % of the ingredient blend. In certain embodiments, the xanthommatin, or a salt thereof, comprises 0.01-1 wt % of the ingredient blend. In certain such embodiments, the xanthommatin, or a salt thereof, comprises 0.4 wt % of the ingredient blend. In certain embodiments, the xanthommatin, or a salt thereof, is ammonium xanthommatin.

In certain embodiments, the effect on one or more sign of skin-aging comprises the reduction, elimination, or slowing of the appearance of wrinkles. In other embodiments, wherein the effect on one or more sign of skin-aging comprises repairing or restoring skin elasticity or firmness. In still other embodiments, the effect on one or more sign of skin-aging comprises reduction, prevention, or slowing the appearance of hyperpigmentation. In certain embodiments, the effect on one or more sign of skin-aging comprises the reduction, slowing or elimination of dysplasias.

In certain embodiments, the ingredient blend includes a rheology modifier. In certain such embodiments, the rheology modifier comprises 0.1-1 wt % (e.g., 0.6 wt %) of the ingredient blend. In certain embodiments, the rheology modifier is carbomer.

In certain embodiments, the ingredient blend includes a humectant. In certain such embodiments, the humectant comprises 0.5-5 wt % (e.g., 3.5 wt %) of the ingredient blend. In certain embodiments, the humectant is glycerin.

In certain embodiments, the ingredient blend includes a preservative. In certain such embodiments, the preservative comprises 0.1-1 wt % (e.g., 0.95 wt %) of the ingredient blend. In certain embodiments, the preservative is phenoxyethanol.

In certain embodiments, the ingredient blend includes a pH adjuster. In certain such embodiments, the pH adjuster comprises 0.1-1 wt % (e.g., 0.4 wt %) of the ingredient blend. In certain embodiments, the pH adjuster is sodium hydroxide.

In implementations, the ingredient blend is suitable for topical administration. The ingredient blend may be in the form of a gel or liquid.

In certain embodiments, the one or more further cosmetic composition comprises one or more moisturizing agent, surfactant, UV absorbing agent, fragrance, anti-oxidant, body pigment, color pigments, or any other suitable general cosmetic composition. In certain embodiments, the one or more further cosmetic composition comprises one or more UV absorbing agent. In certain such embodiments, the one or more UV-absorbing compound agent is selected from avobenzone, oxybenzone, oxybenzone cinoxate, homosalate, octisalate, octinoxate, octocrylene, trolamine salicylate, bemotrizinol, and bisoctrizole. In other such embodiments, the one or more UV-absorbing agent is selected from kaolin, talc, petrolatum and metal oxides. In certain embodiments, the one or more further cosmetic composition comprises one or more antioxidants. In certain such embodiments, the one or more antioxidants is selected from acetyl cysteine, alpha-lipoic acid, arbutin, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, caffeic acid, carotenoids, chlorogenic acid, cysteine, cysteine HCl, diamyihydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, flavone, flavanone, flavanol, gallic acid esters, glutathione, goosypol, hydroquinone, hydroxyanisole, isoflavones, isooctyl thioglycolate, ithiothreitol, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, protocatechuic acid, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thiodipropionic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tris(nonylphenyl)phosphite, Trolox, and xanthines. In certain embodiments, the one or more further cosmetic composition comprises one or more agent selected from alpha and beta hydroxy acids, amino acids, peptides, matrix proteins, growth factors, stem cell activators, estrogens, anti-androgens, and skin lightening and brightening agents.

In certain embodiments, the one or more topical dermatologic drug composition is selected from anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including eflornithine and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, and wound healing agents.

The present application further provides a method of preparing a cosmetic or topical dermatologic composition for preventing or reducing the effects of skin aging in a subject, comprising combining an ingredient blend as disclosed herein and one or more further general cosmetic composition or topical dermatologic drug composition.

The present application further provides a method of preventing or reducing the effects of skin aging in a subject, comprising administering to the subject a cosmetic composition comprising an ingredient blend as disclosed herein combined with one or more further general cosmetic composition or topical dermatologic drug composition.

The present application further provides a method of stimulating collagen production in the skin of a subject, comprising administering to the subject a cosmetic composition comprising an ingredient blend as disclosed herein combined with one or more further general cosmetic composition or topical dermatologic drug composition.

The present application further provides a method of reducing, preventing, or slowing collagen degradation in the skin of a subject, comprising administering to the subject a cosmetic composition comprising an ingredient blend as disclosed herein combined with one or more further general cosmetic composition or topical dermatologic drug composition.

The present application further provides a method of stimulating keratin growth in the skin of a subject, comprising administering to the subject a cosmetic composition comprising an ingredient blend as disclosed herein combined with one or more further general cosmetic composition or topical dermatologic drug composition.

The present application further provides a method of stimulating collagen production and keratin growth in the skin of a subject, comprising administering to the subject a cosmetic composition comprising an ingredient blend as disclosed herein combined with one or more further general cosmetic composition or topical dermatologic drug composition.

The present application further provides a method of reducing, preventing, or slowing the appearance of hyperpigmentation in the skin of a subject, comprising administering to the subject a cosmetic composition comprising an ingredient blend as disclosed herein combined with one or more further general cosmetic or topical dermatologic drug ingredient.

### BRIEF DESCRIPTION OF THE DRAWINGS

This patent or application filed contains at least one drawing executed in color. Copies of this patent or patent application publication with color drawing(s) will be provided by the Office upon request and payment of the necessary fee.

FIG. 1 depicts FTIR spectra of formulation with no xanthommatin (gray), with 0.1% of xanthommatin (blue), and with 0.4% of Xanthommatin (red) as well as on the raw xanthommatin (black).

FIG. 2 depicts the results of the skin penetration studies with a xanthommatin formulation. Content of xanthommatin is depicted on a color scale which would be difficult to discern in black-and-white images.

FIG. 3 depicts the results from the GeneMarkers study in which xanthommatin upregulates key skin markers.

### DETAILED DESCRIPTION

The present application provides an ingredient blend for use in an anti-aging product comprising an unsaturated solution of a phenoxazone and/or phenoxazine compound, or a salt thereof (e.g., xanthommatin, or a salt thereof, such as ammonium xanthommatin), wherein the composition provides an effect on one or more sign of skin-aging. For example, the present application provides an ingredient blend for use in an anti-aging product comprising an unsaturated solution of a phenoxazone and/or phenoxazine compound, or a salt thereof (e.g., xanthommatin, or a salt thereof, such as ammonium xanthommatin), wherein, upon combination with one or more further general cosmetic composition or topical dermatologic drug composition, the composition provides an effect on one or more sign of skin-aging.

The present application provides an ingredient blend for use in an anti-aging product consisting essentially of an unsaturated solution of a phenoxazone and/or phenoxazine compound, or a salt thereof (e.g., xanthommatin, or a salt thereof, such as ammonium xanthommatin), wherein the composition provides an effect on one or more sign of skin-aging. For example, the present application provides an ingredient blend for use in an anti-aging product consisting essentially of an unsaturated solution of a phenoxazone and/or phenoxazine compound, or a salt thereof (e.g., xanthommatin, or a salt thereof, such as ammonium xanthommatin), wherein, upon combination with one or more further general cosmetic composition or topical dermatologic drug composition, the composition provides an effect on one or more sign of skin-aging.

In certain embodiments, the effect on one or more sign of skin-aging comprises the reduction, elimination, or slowing of the appearance of wrinkles. In certain such embodiments, the ingredient blends of the present application reduce, eliminate, or slow the appearance of wrinkles through the increased production of keratin and/or collagen. In other embodiments, the effect on one or more sign of skin-aging comprises repairing or restoring skin elasticity or firmness. In certain such embodiments, the ingredient blends of the present application repair or restore skin elasticity or firmness through the increased production of keratin and/or collagen. In still further embodiments, the effect on one or more sign of skin-aging comprises reduction, prevention, or slowing the appearance of hyperpigmentation. In certain embodiments, the hyperpigmentation is a result of increased melanin production which produces dark spots (e.g., age spots or freckles) on the skin, such as on the hands and/or face. In certain embodiments, the effect on one or more sign of skin-aging comprises the reduction, slowing or elimination of dysplasias. In certain embodiments, signs of skin aging include, but are not limited to, outward visibly and tactilely perceptible manifestations as well as macro or micro effects due to skin aging. These signs may result from processes which include, but are not limited to, the development of textural discontinuities such as wrinkles and coarse deep wrinkles, fine lines, skin lines, crevices, bumps, large pores (e.g., associated with adnexal structures such as sweat gland ducts, sebaceous glands, or hair follicles), or unevenness or roughness, loss of skin elasticity (loss and/or inactivation of functional skin elastin), sagging (including puffiness in the eye area and jowls), loss of skin firmness, loss of skin tightness, loss of skin recoil from deformation, discoloration (including under eye circles), blotching, sallowness, hyperpigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues (e.g., fat and/or muscle), especially those proximate to the skin. In certain embodiments, skin aging may be influenced by factors including hormonal status and climatic, working, social, and cultural conditions. The effects of aging on the skin are influenced by both intrinsic and extrinsic factors. Similar to other organs, the human skin undergoes progressive functional decline due to the accumulation of molecular damage. Oxidative stress and molecular damage contribute to both intrinsic aging, also called chronological aging and aging as a consequence of environmental factors, also called extrinsic aging. In certain embodiments, the skin aging is a result of extrinsic factors, such as smoking, excessive alcohol, and poor nutrition. In certain embodiments, the skin aging is a result of exposure to UV radiation (e.g., from UV radiation from sunlight). In certain embodiments, the skin aging is a result of exposure to poor air quality (e.g., air pollution, including air polluted with particulate matter, soot, and nitrogen dioxide). As a consequence, aged skin exhibits many differences in comparison to youthful skin and also has a marked susceptibility to dermatologic disorders due to the structural and physiologic changes that occur with time. In certain embodiments, the skin aging may be a result of or influenced by a skin disease. In certain such embodiments, the skin disease is atopic dermatitis, seborrheic dermatitis, seborrheic keratosis, epidermolysis bullosa acquisita, psoriasis, ichthyosis, cutaneous lupus erythematosus, dermatomyositis, scleroderma, chronic acne, chronic cellulites, pruritus and abnormal or defective scar formation in diabetes and premature aging diseases. In other embodiments, the skin disease comprises dysplasias, actinic keratosis, basal cell carinoma, squamous cell carcinoma, melanoma. In certain embodiments, the skin aging may be the result of or influenced by a premature aging disease. In certain such embodiments, the premature aging disease is Hutchinson-Gilford Progeria syndrome (HGPS), Atypical progeria syndromes (APS), Mondibuloacral dysplasia (MAD), Werner syndrome (WS), Bloom syndrome (BS), Rothmund-Thomson syndrome (RTS), Cockayne syndrome (CS), Xeroderma Pigmentosum (XP), Trichothiodystrophy (TTD), Fanconi Anemia (FA), Ataxia telangiectasia (AT) and dyskeratosis congenita (DC).

In certain embodiments of the ingredient blends of the present application, the phenoxazone and/or phenoxazine compound, or a salt thereof, is selected from xanthommatin, decarboxylated xanthommatin, uncyclized xanthommatin, ommatin D, dihydroxy-xanthommatin, rhodommatin, a derivative of any of the foregoing, and a precursor of any of the foregoing, and salts of any of the foregoing. In certain such embodiments, the unsaturated solution of a phenoxazone and/or phenoxazine compound, or a salt thereof, comprises unaggregated molecules.

In certain embodiments of the ingredient blends of the present application, the phenoxazone and/or phenoxazine compound, or a salt thereof, is xanthommatin, or a salt thereof. In certain such embodiments, the phenoxazone and/or phenoxazine compound, or a salt thereof, is ammonium xanthommatin.

In certain embodiments of the ingredient blends of the present application, the ingredient blend is water-based. In certain embodiments of the methods of the present application, the ingredient blend is a one-phase system.

In certain embodiments of the ingredient blends of the present application, the phenoxazone and/or phenoxazine compound, or a salt thereof (e.g., xanthommatin, or a salt thereof, such as ammonium xanthommatin), comprises 0.01-1.0 wt % of the ingredient blend. In certain such embodiments, the phenoxazone and/or phenoxazine compound, or a salt thereof (e.g., xanthommatin, or a salt thereof, such as ammonium xanthommatin), comprises 0.05-1.0 wt %, 0.1-1.0 wt %, 0.1-0.9 wt %, 0.1-0.8 wt %, 0.1-0.7 wt %, 0.1-0.6 wt %, 0.1-0.5 wt %, 0.1-0.4 wt %, 0.2-1 wt %, 0.3-1.0 wt %, 0.4-1.0 wt %, 0.5-1.0 wt %, or 0.5-1.0 wt % of the ingredient blend. In certain such embodiments, the phenoxazone and/or phenoxazine compound, or a salt thereof (e.g., xanthommatin, or a salt thereof, such as ammonium xanthommatin), comprises 0.1-0.4 wt % of the ingredient blend. In certain embodiments, the phenoxazone and/or phenoxazine compound (e.g., xanthommatin, or a salt thereof, such as ammonium xanthommatin), or a salt thereof, comprises 0.05 wt %, 0.1 wt %, 0.2 wt %, 0.3 wt %, 0.4 wt %, 0.5 wt %, 0.6 wt %, 0.7 wt %, 0.8 wt %, 0.9 wt %, or 1.0 wt % of the ingredient blend. In certain embodiments, the phenoxazone and/or phenoxazine compound (e.g., xanthommatin, or a salt thereof, such as ammonium xanthommatin), or a salt thereof, comprises 0.4 wt % of the ingredient blend. In certain embodiments, the ingredient blend comprises 0.0055-0.55 g of the phenoxazone and/or phenoxazine compound (e.g., xanthommatin, or a salt thereof, such as ammonium xanthommatin), or a salt thereof. In certain such embodiments, the ingredient blend comprises 0.01-0.5 g, 0.05-0.4 g, 0.10-0.30 g, 0.15-0.30, 0.20-0.25, or 0.22 g of the phenoxazone and/or phenoxazine compound (e.g., xanthommatin, or a salt thereof, such as ammonium xanthommatin), or a salt thereof.

The present application further provides an ingredient blend for use in an anti-aging product comprising a water-based unsaturated solution of xanthommatin, or a salt thereof, wherein the ingredient blend provides an effect on one or more sign of skin-aging, and wherein the xanthommatin, or a salt thereof, comprises up to 1 wt % of the ingredient blend. For example, the present application provides an ingredient blend for use in an anti-aging product comprising a water-based unsaturated solution of xanthommatin, or a salt thereof, wherein, upon combination with one or more further general cosmetic composition or topical dermatologic drug composition, the ingredient blend provides an effect on one or more sign of skin-aging, and wherein the xanthommatin, or a salt thereof, comprises up to 1 wt % of the ingredient blend. In certain such embodiments, the xanthommatin, or a salt thereof, (e.g., ammonium xanthommatin), comprises 0.01-1.0 wt % of the ingredient blend. In certain such embodiments, the xanthommatin, or a salt thereof, (e.g., ammonium xanthommatin), comprises 0.05-1.0 wt %, 0.1-1.0 wt %, 0.1-0.9 wt %, 0.1-0.8 wt %, 0.1-0.7 wt %, 0.1-0.6 wt %, 0.1-0.5 wt %, 0.1-0.4 wt %, 0.2-1 wt %, 0.3-1.0 wt %, 0.4-1.0 wt %, 0.5-1.0 wt %, or 0.5-1.0 wt % of the ingredient blend. In certain embodiments, the xanthommatin, or a salt thereof, (e.g., ammonium xanthommatin), comprises 0.1-0.4 wt % of the ingredient blend. In certain embodiments, the xanthommatin, or a salt thereof, (e.g., ammonium xanthommatin), comprises 0.05 wt %, 0.1 wt %, 0.2 wt %, 0.3 wt %, 0.4 wt %, 0.5 wt %, 0.6 wt %, 0.7 wt %, 0.8 wt %, 0.9 wt %, or 1.0 wt % of the ingredient blend. In certain embodiments, the xanthommatin, or a salt thereof, (e.g., ammonium xanthommatin), comprises 0.4 wt % of the ingredient blend. In certain embodiments, the ingredient blend comprises 0.0055-0.55 g of the xanthommatin, or a salt thereof, (e.g., ammonium xanthommatin). In certain such embodiments, the ingredient blend comprises 0.01-0.5 g, 0.05-0.4 g, 0.10-0.30 g, 0.15-0.30, 0.20-0.25, or 0.22 g of the xanthommatin, or a salt thereof, (e.g., ammonium xanthommatin).

The present application further provides an ingredient blend for use in an anti-aging product consisting essentially of a water-based unsaturated solution of xanthommatin, or a salt thereof, wherein the ingredient blend provides an effect on one or more sign of skin-aging, and wherein the xanthommatin, or a salt thereof, comprises up to 1 wt % of the ingredient blend. For example, the present application provides an ingredient blend for use in an anti-aging product consisting essentially of a water-based unsaturated solution of xanthommatin, or a salt thereof, wherein, upon combination with one or more further general cosmetic composition or topical dermatologic drug composition, the ingredient blend provides an effect on one or more sign of skin-aging, and wherein the xanthommatin, or a salt thereof, comprises up to 1 wt % of the ingredient blend. In certain such embodiments, the xanthommatin, or a salt thereof, (e.g., ammonium xanthommatin), comprises 0.01-1.0 wt % of the ingredient blend. In certain such embodiments, the xanthommatin, or a salt thereof, (e.g., ammonium xanthommatin), comprises 0.05-1.0 wt %, 0.1-1.0 wt %, 0.1-0.9 wt %, 0.1-0.8 wt %, 0.1-0.7 wt %, 0.1-0.6 wt %, 0.1-0.5 wt %, 0.1-0.4 wt %, 0.2-1 wt %, 0.3-1.0 wt %, 0.4-1.0 wt %, 0.5-1.0 wt %, or 0.5-1.0 wt % of the ingredient blend. In certain embodiments, the xanthommatin, or a salt thereof, (e.g., ammonium xanthommatin), comprises 0.1-0.4 wt % of the ingredient blend. In certain embodiments, the xanthommatin, or a salt thereof, (e.g., ammonium xanthommatin), comprises 0.05 wt %, 0.1 wt %, 0.2 wt %, 0.3 wt %, 0.4 wt %, 0.5 wt %, 0.6 wt %, 0.7 wt %, 0.8 wt %, 0.9 wt %, or 1.0 wt % of the ingredient blend. In certain embodiments, the xanthommatin, or a salt thereof, (e.g., ammonium xanthommatin), comprises 0.4 wt % of the ingredient blend. In certain embodiments, the ingredient blend comprises 0.0055-0.55 g of the xanthommatin, or a salt thereof, (e.g., ammonium xanthommatin). In certain such embodiments, the ingredient blend comprises 0.01-0.5 g, 0.05-0.4 g, 0.10-0.30 g, 0.15-0.30, 0.20-0.25, or 0.22 g of the xanthommatin, or a salt thereof, (e.g., ammonium xanthommatin).

In certain embodiments of the ingredient blends of the present application, the ingredient blend further comprises a rheology modifier. In certain such embodiments, the rheology modifier comprises 0.1-1 wt % of the ingredient blend. In certain embodiments, the rheology modifier comprises 0.1 wt %, 0.2 wt %, 0.3 wt %, 0.4 wt %, 0.5 wt %, 0.6 wt %, 0.7 wt %, 0.8 wt %, 0.9 wt %, or 1.0 wt % of the ingredient blend. In certain embodiments, the rheology modifier comprises 0.6 wt % of the ingredient blend. In certain embodiments, the rheology modifier is present in an amount that prevents significant dripping or pooling of the composition after application to the skin. In certain embodiments, the rheology modifier is carbomer. In some embodiments, the rheology modifier is selected from stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

Additional examples of rheology modifiers include thickener or gelling agents, including substances which that can increase the viscosity of a composition. Thickening agents include those that can increase the viscosity of a composition without substantially modifying the efficacy of the active ingredient within the composition. Thickening agents can also increase the stability of the compositions of the present invention. In certain aspects of the present invention, thickening agents include hydrogenated polyisobutene or trihydroxy stearin, or a mixture of both. Additional non-limiting examples of additional thickening agents that can be used in the context of the present application include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see CTFA International Cosmetic Ingredient Dictionary, Fourth Edition, 1991, pp. 12 and 80). Examples of commercially available carboxylic acid polymers include carbomers, which are homopolymers of acrylic acid crosslinked with allyl ethers of sucrose or pentaerythritol (e.g., Carbopol™ 900 series from B. F. Goodrich). Non-limiting examples of crosslinked polyacrylate polymers include cationic and nonionic polymers.

Non-limiting examples of polyacrylamide polymers (including nonionic polyacrylamide polymers including substituted branched or unbranched polymers) include polyacrylamide, isoparaffin and Laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropyl cellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer are hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present compositions include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof. In one embodiment, the thickening agent is Chondrus crispus (carrageenan) extract.

In certain embodiments of the ingredient blends of the present application, the ingredient blend further comprises a moisturizing agent (e.g., humectant). In certain such embodiments, the moisturizing agent (e.g., humectant) comprises 0.5-5.0 wt % of the ingredient blend. In certain embodiments, the moisturizing agent (e.g., humectant) comprises 1.0-5.0 wt % of the ingredient blend. In certain embodiments, the moisturizing agent (e.g., humectant) comprises 1.5-5.0 wt %, 2.0-5.0 wt %, 2.0-4.0 wt %, 2.5-3.5 wt %, or 3.0-4.0 wt % of the ingredient blend. In certain embodiments, the moisturizing agent (e.g., humectant) comprises 3.5 wt % of the ingredient blend. In certain embodiments, the humectant is glycerin.

Additional examples of moisturizing agents that can be used with the ingredient blend of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydroly sate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol. In one embodiment, the moisturizing agent is glycerin. Other examples include acetylated lanolin, acetylated lanolin alcohol, alanine, algae extract, aloe barbadensis, aloe-barbadensis extract, aloe barbadensis gel, althea officinalis extract, apricot (Prunus armeniaca) kernel oil, arginine, arginine aspartate, arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, calendula officinalis extract, calendula officinalis oil, candelilla (Euphorbia cerifera) wax, canola oil, caprylic/capric triglyceride, cardamon (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (Oenothera biennis) oil, fatty acids, geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil. Additional non-limiting examples of moisturizing agents may include PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinol palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Trificum vulgare) germ oil, and ylang ylang (Cananga odorata) oil. In one embodiment, the moisturizing agent may be allantoin.

In certain embodiments of the ingredient blends of the present application, the ingredient blend further comprises a preservative. In certain such embodiments, the preservative comprises 0.1-2 wt % of the ingredient blend. In certain such embodiments, the preservative comprises 0.1-1 wt % of the ingredient blend. In certain such embodiments, the preservative comprises 0.5-1 wt % of the ingredient blend. In certain embodiments, the preservative comprises 0.65 wt %, 0.7 wt %, 0.75 wt %, 0.8 wt %, 0.85 wt %, 0.9 wt %, or 1.0 wt % of the ingredient blend. In certain embodiments, the preservative comprises 0.95 wt % of the ingredient blend. In certain embodiments, the preservative is phenoxyethanol. In certain embodiments, the preservative is selected from one or more of quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, ethylhexylglycerin, ethylhexylglycerinbenzyl alcohol, chlorobutanol, phenol, sorbic acid, thimerosal and combinations thereof.

In certain embodiments of the ingredient blends of the present application, the ingredient blend further comprises a pH adjuster. In certain such embodiments, the pH adjuster comprises 0.1-1 wt % of the ingredient blend. In certain embodiments, the pH adjuster comprises 0.1 wt %, 0.2 wt %, 0.3 wt %, 0.4 wt %, 0.5 wt %, 0.6 wt %, 0.7 wt %, 0.8 wt %, 0.9 wt %, or 1.0 wt % of the ingredient blend. In certain embodiments, the pH adjuster comprises 0.4 wt % of the ingredient blend. In certain embodiments, the pH adjuster is sodium hydroxide.

The present application further provides an ingredient blend for use in an anti-aging product comprising 1) a water-based unsaturated solution of xanthommatin, or a salt thereof (e.g., ammonium xanthommatin), 2) a rheology modifier (e.g., carbomer), 3) a humectant (e.g., glycerin), 4) a preservative (e.g., phenoxyethanol), and 5) a pH adjuster (e.g., sodium hydroxide), wherein the ingredient blend provides an effect on one or more sign of skin-aging, and wherein the xanthommatin, or a salt thereof, comprises up to 1 wt % of the ingredient blend. For example, the present application provides an ingredient blend for use in an anti-aging product comprising 1) a water-based unsaturated solution of xanthommatin, or a salt thereof (e.g., ammonium xanthommatin), 2) a rheology modifier (e.g., carbomer), 3) a humectant (e.g., glycerin), 4) a preservative (e.g., phenoxyethanol), and 5) a pH adjuster (e.g., sodium hydroxide), wherein, upon combination with one or more further general cosmetic composition or topical dermatologic drug composition, the ingredient blend provides an effect on one or more sign of skin-aging, and wherein the xanthommatin, or a salt thereof, comprises up to 1 wt % of the ingredient blend. In certain such embodiments, the xanthommatin, or a salt thereof, (e.g., ammonium xanthommatin), comprises 0.01-1.0 wt % of the ingredient blend. In certain embodiments, the xanthommatin, or a salt thereof, (e.g., ammonium xanthommatin), comprises 0.4 wt % of the ingredient blend. In certain embodiments, the rheology modifier (e.g., carbomer) comprises 0.1-1 wt % of the ingredient blend, such as 0.6 wt % of the ingredient blend. In certain embodiments, the humectant (e.g., glycerin) comprises 0.5-5 wt % of the ingredient blend, such as 3.5 wt % of the ingredient blend. In certain embodiments, the preservative (e.g., phenoxyethanol) comprises 0.1-1 wt % of the ingredient blend, such as 0.95 wt % of the ingredient blend. In certain embodiments, the pH adjuster (e.g., sodium hydroxide) comprises 0.1-1 wt % of the ingredient blend, such as 0.4 wt % of the ingredient blend. In certain embodiments, the present application provides an ingredient blend for use in an anti-aging product comprising 1) a water-based unsaturated solution of xanthommatin, or a salt thereof (e.g., ammonium xanthommatin), 2) 0.1-1 wt % (e.g., 0.6 wt %) of a rheology modifier (e.g., carbomer), 3) 0.5-5 wt % (e.g., 3.5 wt %) of a humectant (e.g., glycerin), 4) 0.1-1 wt % (e.g., 0.95 wt %) of a preservative (e.g., phenoxyethanol), and 5) 0.1-1 wt % (e.g., 0.4 wt %) of a pH adjuster (e.g., sodium hydroxide), wherein, upon combination with one or more further general cosmetic composition or topical dermatologic drug composition, the ingredient blend provides an effect on one or more sign of skin-aging, and wherein the xanthommatin, or a salt thereof, comprises up to 1 wt % of the ingredient blend, such as 0.01-1.0 wt % (e.g., 0.4 wt %) of the ingredient blend.

The present application further provides an ingredient blend for use in an anti-aging product consisting essentially of 1) a water-based unsaturated solution of xanthommatin, or a salt thereof (e.g., ammonium xanthommatin), 2) a rheology modifier (e.g., carbomer), 3) a humectant (e.g., glycerin), 4) a preservative (e.g., phenoxyethanol), and 5) a pH adjuster (e.g., sodium hydroxide), wherein the ingredient blend provides an effect on one or more sign of skin-aging, and wherein the xanthommatin, or a salt thereof, comprises up to 1 wt % of the ingredient blend. For example, the present application provides an ingredient blend for use in an anti-aging product consisting essentially of 1) a water-based unsaturated solution of xanthommatin, or a salt thereof (e.g., ammonium xanthommatin), 2) a rheology modifier (e.g., carbomer), 3) a humectant (e.g., glycerin), 4) a preservative (e.g., phenoxyethanol), and 5) a pH adjuster (e.g., sodium hydroxide), wherein, upon combination with one or more further general cosmetic composition or topical dermatologic drug composition, the ingredient blend provides an effect on one or more sign of skin-aging, and wherein the xanthommatin, or a salt thereof, comprises up to 1 wt % of the ingredient blend. In certain such embodiments, the xanthommatin, or a salt thereof, (e.g., ammonium xanthommatin), comprises 0.01-1.0 wt % of the ingredient blend. In certain embodiments, the xanthommatin, or a salt thereof, (e.g., ammonium xanthommatin), comprises 0.4 wt % of the ingredient blend. In certain embodiments, the rheology modifier (e.g., carbomer) comprises 0.1-1 wt % of the ingredient blend, such as 0.6 wt % of the ingredient blend. In certain embodiments, the humectant (e.g., glycerin) comprises 0.5-5 wt % of the ingredient blend, such as 3.5 wt % of the ingredient blend. In certain embodiments, the preservative (e.g., phenoxyethanol) comprises 0.1-1 wt % of the ingredient blend, such as 0.95 wt % of the ingredient blend. In certain embodiments, the pH adjuster (e.g., sodium hydroxide) comprises 0.1-1 wt % of the ingredient blend, such as 0.4 wt % of the ingredient blend. In certain embodiments, the present application provides an ingredient blend for use in an anti-aging product consisting essentially of 1) a water-based unsaturated solution of xanthommatin, or a salt thereof (e.g., ammonium xanthommatin), 2) 0.1-1 wt % (e.g., 0.6 wt %) of a rheology modifier (e.g., carbomer), 3) 0.5-5 wt % (e.g., 3.5 wt %) of a humectant (e.g., glycerin), 4) 0.1-1 wt % (e.g., 0.95 wt %) of a preservative (e.g., phenoxyethanol), and 5) 0.1-1 wt % (e.g., 0.4 wt %) of a pH adjuster (e.g., sodium hydroxide), wherein, upon combination with one or more further general cosmetic composition or topical dermatologic drug composition, the ingredient blend provides an effect on one or more sign of skin-aging, and wherein the xanthommatin, or a salt thereof, comprises up to 1 wt % of the ingredient blend, such as 0.01-1.0 wt % (e.g., 0.4 wt %) of the ingredient blend.

In certain embodiments, the ingredient blends of the present application may further comprise excipients commonly used in the formulation of cosmetic or pharmaceutical preparations for topical use, such as bactericidal agents, stabilizers, emulsifiers, buffers, wetting agents, coloring agents, and other excipients commonly used in the cosmetic/pharmaceutical preparation techniques.

In certain embodiments of the ingredient blends of the present application, the ingredient blend further comprises one or more emulsifiers. In certain such embodiments, the emulsifier reduces the interfacial tension between phases and improves the formulation and stability of an emulsion. The emulsifier may include a non-ionic emulsifier, an anionic emulsifier, a cationic emulsifier, a Zwitterionic emulsifier or a combination thereof. Non-limiting examples of emulsifiers include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, Steareth-2, Steareth-20, Steareth-21, ceteareth-20. PPG-2 methylglucose ether distearate, ceteth 10, polysorbate 80, cetyl phosphate, potassium cetyl phosphate, diethanolamine cetyl phosphate, polysorbate 60, glyceryl Stearate, PEG-100 stearate, and mixtures thereof. In one embodiment, the non-ionic emulsifier is cetearyl olivate or sorbitan olivate.

In certain embodiments, the ingredient blends of the present application are combined with one or more further cosmetic composition prior to use.

In certain embodiments of the present application wherein the ingredient blend is combined with one or more further cosmetic composition prior to use, the one or more further cosmetic composition comprises one or more moisturizing agent, surfactant, UV absorbing agent, fragrance, anti-oxidant, preservative, body pigment, color pigments pH adjusting agent, solvent or any other suitable general cosmetic or topical dermatologic drug ingredient.

In certain embodiments of the present application wherein the ingredient blend is combined with one or more further cosmetic composition prior to use, the one or more further cosmetic composition comprises one or more UV absorbing agent. In certain such embodiments, the one or more UV-absorbing agent is selected from avobenzone, oxybenzone, oxybenzone cinoxate, homosalate, octisalate, octinoxate, octocrylene, and trolamine salicylate, bemotrizinol, and bisoctrizole. In certain embodiments, the UV-absorbing agent includes chemical and physical sunblocks. Non-limiting examples of chemical sunblocks that can be used include bemotrizinol (Tinosorb S), bisoctrizole (Tinosorb M), para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. Non-limiting examples of physical sunblocks include kaolin, talc, petrolatum and metal oxides (e.g., titanium dioxide and zinc oxide).

In certain embodiments of the present application wherein the ingredient blend is combined with one or more further cosmetic composition prior to use, the one or more further cosmetic composition comprises one or more antioxidant. Non-limiting examples of antioxidants that can be used with the compositions of the present invention include acetyl cysteine, alpha-lipoic acid, arbutin, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, caffeic acid, carotenoids, chlorogenic acid, cysteine, cysteine HCl, diamyihydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, flavone, flavanone, flavanol, gallic acid esters, glutathione, goosypol, hydroquinone, hydroxyanisole, isoflavones, isooctyl thioglycolate, ithiothreitol, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, protocatechuic acid, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thiodipropionic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tris(nonylphenyl)phosphite, Trolox, and xanthines.

In certain embodiments of the present application wherein the ingredient blend is combined with one or more further cosmetic composition prior to use, the one or more further cosmetic composition comprises one or more agent selected from alpha and beta hydroxy acids, amino acids, peptides, matrix proteins, growth factors, stem cell activators, estrogens, anti-androgens, and skin lightening and brightening agents.

In certain embodiments of the present application wherein the ingredient blend is combined with one or more further cosmetic composition prior to use, the one or more further cosmetic composition comprises one or more cosmetic ingredient. A wide variety of non-limiting cosmetic ingredients described in the CTFA International Cosmetic Ingredient Dictionary and Handbook (2004 and 2008) can be used. Non-limiting examples of cosmetic ingredients include fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraminobenzoic acid (“PABA”) and corresponding PABA derivatives, titanium dioxide, zinc oxide, etc.), essential oils, vitamins (e.g., A, B, C, D, E, and K), trace metals (e.g. zinc, calcium and selenium), anti-irritants (e.g. steroids and non-steroidal anti-inflammatoireotanical extracts (e.g. aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate).

In certain embodiments of the present application, the ingredient blend is combined with one or more topical dermatologic drug composition prior to use. In certain such embodiments, the pharmaceutically active agent is selected from anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including eflornithine and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

In certain embodiments of the ingredient blends of the present application, the composition (e.g., the ingredient blend itself and/or the ingredient blend in combination with one or more further cosmetic composition or topical dermatologic drug composition) is suitable for topical administration. In certain such embodiments, the ingredient blends of the present application are suitable for topical application on the skin (e.g., on the face or hands). In certain such embodiments, the ingredient blends of the present application are suitable for topical application on the scalp. In certain embodiments of the methods and ingredient blends of the present application, skin refers to the epidermis, the dermis and the subcutis, such as the epidermis and the dermis (e.g., the epidermis). As used herein, “epidermis” refers to the outer layer of skin, and is divided into five strata, which include the: stratum corneum, stratum lucidum, stratum granulosum, stratum spinosum, and stratum basale. The stratum corneum contains many layers of dead, anucleated keratinocytes that are essentially filled with keratin. In certain embodiments, the ingredient blends of the present application are dermatologically-acceptable (e.g., they do not have undue toxicity, incompatibility, instability, allergic response, and the like, when applied to the skin).

In certain embodiments of the present application wherein the ingredient blend is combined with a further cosmetic composition or topical dermatologic drug composition prior to use, the final composition is applied to the affected skin area one or more time per day.

In certain embodiments of the ingredient blends of the present application, the ingredient blend is in the form of a gel or liquid. In certain such embodiments, the ingredient blend is in the form of a gel. In certain embodiments of the present application wherein the ingredient blend is combined with a further cosmetic composition or topical dermatologic drug composition prior to use, the final composition is in the form of a lotion, a cream, a gel, a emulsion (e.g., oil-in-water, water-in-oil, silicone-in water, water-in-silicone, water-in-oil-in-water, oil-in-water-in-oil, oil-in-water-in-silicone, etc.), solutions (e.g., aqueous or hydro-alcoholic solutions), anhydrous bases (e.g., lipstick or a powder), ointments, milk, paste, aerosol, solid forms, eye jellies, or powdered form (e.g., dried, lyophilized, particulate, etc.).

### Methods

The present application further provides a method of preparing a cosmetic or topical dermatologic composition for preventing or reducing the effects of skin aging in a subject, comprising combining an ingredient blend as disclosed herein and one or more further general cosmetic composition or topical dermatologic drug composition.

The present application further provides a method of preventing or reducing the effects of skin aging in a subject, comprising administering to the subject a cosmetic composition comprising an ingredient blend as disclosed herein (e.g., an unsaturated solution of a phenoxazone and/or phenoxazine compound, or a salt thereof, such as xanthommatin, or a salt thereof, (e.g., ammonium xanthommatin)), combined with one or more further general cosmetic composition or topical dermatologic drug composition.

The present application further provides a method of stimulating collagen production in the skin of a subject, comprising administering to the subject a cosmetic composition comprising an ingredient blend as disclosed herein (e.g., an unsaturated solution of a phenoxazone and/or phenoxazine compound, or a salt thereof, such as xanthommatin, or a salt thereof, (e.g., ammonium xanthommatin)) combined with one or more further general cosmetic composition or topical dermatologic drug composition.

The present application further provides a method of reducing, preventing, or slowing collagen degradation in the skin of a subject, comprising administering to the subject a cosmetic composition comprising an ingredient blend as disclosed herein (e.g., an unsaturated solution of a phenoxazone and/or phenoxazine compound, or a salt thereof, such as xanthommatin, or a salt thereof, (e.g., ammonium xanthommatin)) combined with one or more further general cosmetic composition or topical dermatologic drug composition.

The present application further provides a method of stimulating keratin growth in the skin of a subject, comprising administering to the subject a cosmetic composition comprising an ingredient blend as disclosed herein (e.g., an unsaturated solution of a phenoxazone and/or phenoxazine compound, or a salt thereof, such as xanthommatin, or a salt thereof, (e.g., ammonium xanthommatin)) combined with one or more further general cosmetic composition or topical dermatologic drug composition.

The present application further provides a method of stimulating collagen production and keratin growth in the skin of a subject, comprising administering to the subject a cosmetic composition comprising an ingredient blend as disclosed herein (e.g., an unsaturated solution of a phenoxazone and/or phenoxazine compound, or a salt thereof, such as xanthommatin, or a salt thereof, (e.g., ammonium xanthommatin)) combined with one or more further general cosmetic composition or topical dermatologic drug composition.

The present application further provides a method of reducing, preventing, or slowing the appearance of hyperpigmentation in the skin of a subject, comprising administering to the subject a cosmetic composition comprising an ingredient blend as disclosed herein (e.g., an unsaturated solution of a phenoxazone and/or phenoxazine compound, or a salt thereof, such as xanthommatin, or a salt thereof, (e.g., ammonium xanthommatin)) combined with one or more further general cosmetic composition or topical dermatologic drug composition.

In certain embodiments of the methods and cosmetic compositions of the present application, the subject is a human subject.

The foregoing is merely illustrative of the principles of the disclosure, and the apparatuses can be practiced by other than the described implementations, which are presented for purposes of illustration and not of limitation. Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems, composition, and formulations. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

### Exemplification

The invention now being generally described, it will be more readily understood by reference to the following examples referring to the results documented in Appendix A, which are included merely for purposes of illustration of certain aspects and embodiments of the present invention, and are not intended to limit the invention.

### Example 1: Skin Penetration Studies

This study was conducted to evaluate and visualize the penetration of 0.1% xanthommatin and 0.4% xanthommatin formulations inside human skin sample. Specifically, the 0.1% xanthommatin contains 0.1% ammonium xanthommatin (Xa), 90% water, 3.5% glycerin, 0.6% carbomer, 0.4% sodium hydroxide, and 0.95% phenoxyethanol. The 0.4% xanthommatin contains 0.4% ammonium xanthommatin (Xa), 90% water, 3.5% glycerin, 0.6% carbomer, 0.4% sodium hydroxide, and 0.95% phenoxyethanol.

Substrate. Human skin was purchased from a licensed supplier. The skin samples used for this project were from the same skin batch.

Experimental protocol for FTIR imaging measurement.
1. Flash-frozen human skin from the same donor purchased from licensed supplier was used for all FTIR experiments.
2. 2.5 cm×2.5 cm piece of skin was cut, thawed and cleaned.
3. Skin was tape-stripped two times to clean the surface and imaged by ATR-FTIR to record control images.
4. 40 mg of the formulation provided by the sponsor was applied on the skin. Product was massaged on the skin using a glass rod and allowed to sit for 5 mins.
5. Skin was placed on a Franz diffusion cell for 4 hours at 32° C.
6. After 4 hours, any excess product on the skin surface was gently blotted with a Kim wipe.
7. Skin was imaged by ATR-FTIR to evaluate initial product deposition on the skin surface. In order to investigate the penetration inside the SC, 8 tape strips were applied and removed from the skin surface. 6 Different depths inside the SC were obtained through this tape stripping method. This method allowed us to image the surface of the SC as well as 5 layers under the surface. After tape strip 1, 2, 4, 6 and 8.

ATR-FTIR imaging spectroscopy was used to scan the skin surfaces before and after sequential tape stripping. The ATR-FTIR images were recorded with a Spotlight system (Perkin Elmer).
Spectral parameters used for the ATR-FTIR imaging measurements
System: Spotlight 400 (PerkinElmer) with ATR-FTIR imaging accessory
Spectral Resolution: 4 cm−1
Spatial Resolution: 6.25 μm
Scan accumulation: 4
Spectral Range: 4000-750 cm−1
All the ATR-FTIR images were concatenated and baseline corrected.
2 skin areas were investigated at each layer inside the stratum corneum.

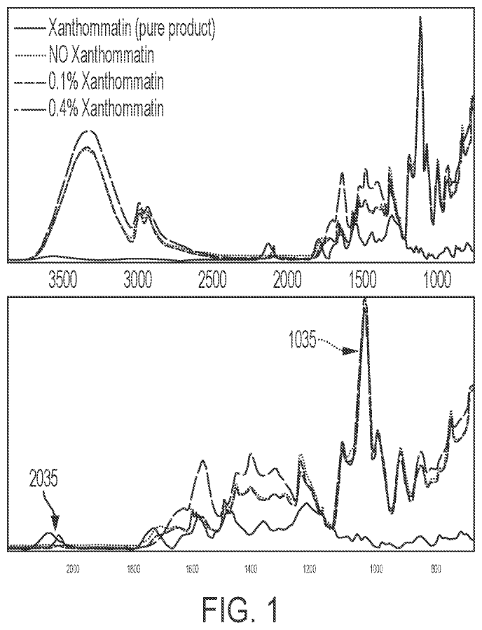

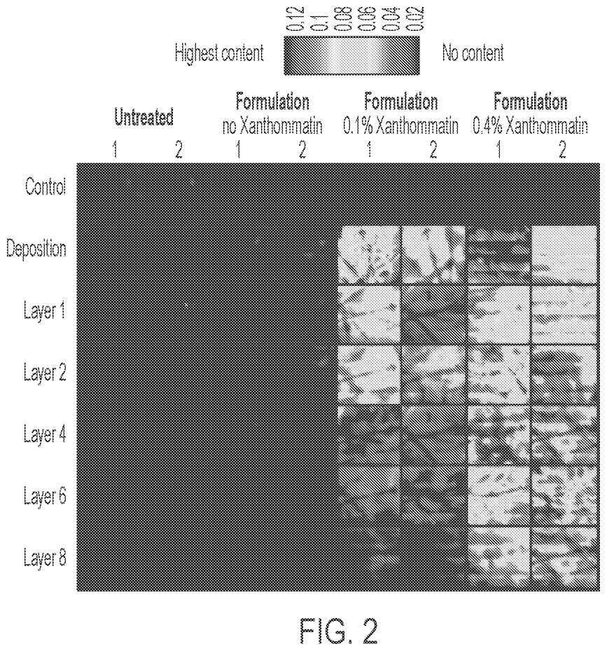

FIG. 1 shows typical FTIR spectra of formulation with no Xanthommatin (gray), with 0.1% of Xanthommatin (blue), and with 0.4% of Xanthommatin (red) as well as on the raw Xanthommatin (black). Two IR markers were used to investigate the penetration of the formulations provided by the sponsor for this project. The band at 2035 cm−1 was selected to investigate specifically the penetration of the Xanthommatin into human skin. Indeed, there is no overlapping in this area with the skin contribution. The band around 1035 cm⁻¹ was selected to investigate the penetration of the Formulation developed by the sponsor into human skin. To visualize the Xanthommatin delivery inside the stratum corneum, specific ATR-FTIR images were generated. FIG. 2 the distribution of the Xanthommatin based on 2035 cm⁻¹ band area into untreated human skin samples (control), untreated skin sample (untreated) and skin samples treated with the 0.1% and 0.4% xanthommatin formulations. Two skin areas were investigated at each layer. The higher the value, the redder the image and the higher xanthommatin content inside the human stratum corneum.

As expected, no Xanthommatin was detected in all untreated human skin samples (control). After 4-hour treatment time in the Franz cell, no penetration was detected in the untreated skin sample or in the skin sample treated with the formulation with no Xanthommatin whatever the layer investigated inside the human stratum corneum. In contrast, after 4-hour treatment time in the Franz cell, Xanthommatin was strongly detected at the surface of the human skin samples treated with formulations with Xanthommatin (0.1 and 0.4%). The formulations tested allowed delivery of Xanthommatin deep inside the human stratum corneum. Indeed, the Xanthommatin was detected up to the layer 8 in the skin treated with the formulation with 0.4% of Xanthommatin. The Xanthommatin delivery was more limited in the skin treated with the formulation with 0.1% of Xanthommatin.

### Example 2: Gene Expression Assay

This study was conducted to determine how different concentrations (0.316 μM-10.0 mM) of xanthommatin influences gene expression in ex vivo NativeSkin® human skin tissues (purchased from Genoskin). NativeSkin® human skin models hold normal skin barrier function, a mature stratum corneum, a functional basal layer and all cell types and skin appendages of in vivo human skin.

Gene expression was assessed using Genemarkers Standard Skin Panel, a qPCR-based gene expression panel which contains 107 target genes potentially relevant in skin biology and 5 endogenous control genes. Gene expression was assessed 24 hrs following application of the test material to the skin tissues.

WST-8 viability assay was performed with a portion of each treated tissue to ensure no adverse effects of the test material on tissue viability. Cell viability of the material and vehicle groups (buffered MES) were compared to an Untreated Control Group. Tissues treated with Triton X-100 were used as the negative control. The test material did not produce any significant reduction in cell viability, as determined using a WST-8 viability assay.

Formulation design. A gel containing either 0.1% or 0.4% (Table 1) by mass percentage of ammonium xanthommatin (Xa) was prepared. Phase A was initiated by adding 0.3 wt % of carbomer into the water without stirring. Next, the solution was heated to 75° C. with mixing to allow the carbomer to disperse throughout the mixture before ammonium xanthommatin was slowly added and allowed to mix for full dissolution. In a separate reaction vessel, Phase B was created with glycerin (3.5%) and phenoxyethanol (0.95%) and mixed well. Phase B was then added to Phase A. The final phase (Phase C), containing 4% deionized water and 0.4% sodium hydroxide, was added to the main phase for neutralization. The gel was then cooled to room temperature.

TABLE 1

| | Ingredient | | Weight | Actual |
|---|---|---|---|---|
| Phase | Name | Functionality | % | Weight |
| A | Deionized | | 90.150 | 207.345 |
| | Water | | | |
| A | Carbomer | Rheology | 0.600 | 1.38 |
| | | Modifier | | |
| A | ammonium | Anti-aging | 0.400 | 0.92 |
| | xanthommatin | | | |
| B | Glycerin | Humectant | 3.500 | 8.05 |
| B | Phenoxyethanol | Preservative | 0.950 | 2.185 |
| C | Deionized | | 4.000 | 9.2 |
| | Water | | | |
| C | Sodium | pH Adjuster | 0.400 | 0.92 |
| | Hydroxide 30% | | | |
| | Total | | 100.000 | 230 |

Tissue equilibration. Native tissues (Genoskin) were equilibrated overnight at 37° C. with 5% CO₂ and ˜95% relative humidity. The following day, the equilibrium medium was removed from each well and replaced with 1.0 mL of maintenance medium.

Treatment Media. Tissues were obtained from the abdomen of a 36-year old Hispanic female with Fitzpatrick type 2-3 skin. Genoskin facilities mounted 11 mm biopsies into Nativeskin tissue inserts which were used for genetic analysis 5 days later. N=4 biological replicates per treatment group. For WST-8 (−) control tissue (N=2), 10 μL of Triton X-100 (1%) was applied to the surface of the tissue; two untreated tissues served as the WST-8 control.

Tissue and medium collections. A 2 mm biopsy punch was taken from the center for the WST-8 assay. Remaining tissues were placed in a tube containing RNAlater preservative solutions. Tissues were incubated for 1-2 hrs until RNA was isolated. RNA was isolated using an RNeasy Mini kit (Qiagen) following the manufacturer's instructions. Each sample was vacuum concentrated to at least 200 ng/μL to provide sufficient amount of RNA for cDNA synthesis. RNA concentration and purity were determined using a Nanodrop 2000 spectrophotometer.

cDNA was generated using a High Capacity cDNA Synthesis Kit (Applied Biosystems) and was generated from 2000 ng RNA per sample for OpenArray processing. qPCR data quality and statistical analysis was assessed and performed on the raw data files using ThermoFisher Connect Software (Life Technologies). Statistical analysis was performed using the relative quantitation (RQ) method, where the Cq value of the target gene is normalized to the Cq value of an endogenous control gene (selected from one of GAPDH, GUSB, HPRT1, PPIA, and UBC) to generate the delta Cq values, which were used to normalize variability between the samples.

Statistical analysis. Unpaired t-tests were carried out using the ThermoFisher Connect software.

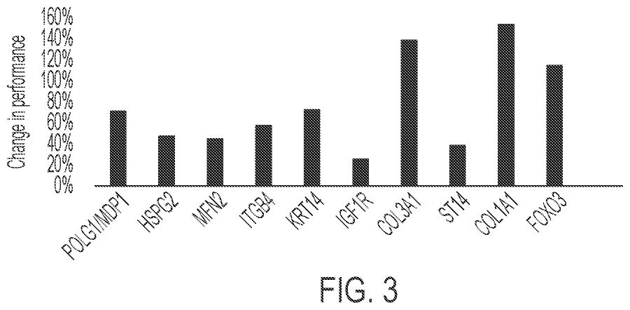

Results. Table 2 lists the statistically significant (p<0.05, N=4) changes in upregulated gene expression for the 10 mM (0.4%) and 3.16 mM (0.1%) dose groups. The linear fold change values (FC) values <1.44 are not statistically significant. As is shown, a 72%, 137%, and 153% increase in gene markers related to Keratin 14 (KRT14), collagen (COL3A1), and COL1A1, respectively, was observed for the 10.0 mM (0.4%) ammonium xanthommatin formulation. These genes play important roles in maintaining healthy skin. For the 3.16 mM (0.1%) ammonium xanthommatin formulation, a 74% upregulation of hyaluronan synthase, an enzyme that is necessary to produce hyaluronan in the extracellular matrix, was observed. FIG. 3 provides an illustration of the statistically significant percentage change in expression for genes with the 0.4% (i.e., 10 mM) ammonium xanthommatin formulation.

TABLE 2

| | | | | | | % |
|---|---|---|---|---|---|---|
| Dose | Gene | | Rq | P-Value | FC | Change |
| 10 mM | POLG1/MDP1 | DNA Polymerase Gamma | 1.713 | 0.04 | 1.71 | 71% |
| 10 mM | HSPG2 | Heparan Sulfate | 1.477 | 0.04 | 1.48 | 48% |
| | | Proteoglycan 2 | | | | |
| 10 mM | MFN2 | mitofusin 2 | 1.447 | 0.02 | 1.45 | 45% |
| 10 mM | ITGB4 | Integrin alpha-6/beta-4 is a | 1.582 | 0.04 | 1.58 | 58% |
| | | receptor for laminin | | | | |
| 10 mM | KRT14 | keratin 14 | 1.721 | 0.03 | 1.72 | 72% |
| 10 mM | IGF1R | insulin-like growth factor | 1.257 | 0.04 | 1.26 | 26% |
| | | 1 | | | | |
| 10 mM | COL3A1 | collagen | 2.37 | 0.02 | 2.37 | 137% |
| 10 mM | ST14 | Suppressor of | 1.385 | 0.03 | 1.39 | 39% |
| | | tumorigenicity 14 protein | | | | |
| 10 mM | COL1A1 | collagen | 2.525 | 0.01 | 2.53 | 153% |
| 10 mM | FOXO3 | subclass of | 2.142 | 0.03 | 2.14 | 114% |
| | | the forkhead family | | | | |
| | | of transcription factors | | | | |
| 3.16 mM | HAS2 | Hyaluronan synthase 2 | 1.74 | 0.04 | 1.74 | 74% |

### Example 3: Skin Irritation and Sensitization Studies in a Human Repeat Insult Patch Test

This single blind study was conducted to evaluate the irritation and sensitization potential of 0.0% ammonium xanthommatin and 0.4% ammonium xanthommatin formulations on healthy volunteers by means of repeated cutaneous occlusive patch applications based on the modified Draize method of Jordan and King (Jordan, W. P. et al., Contact Dermatitis, 3 (1977) 19-26). Specifically, the 0.0% xanthommatin contains 0.0% ammonium xanthommatin (Xa), 90.4% water, 3.5% glycerin, 0.6% carbomer, 0.4% sodium hydroxide, and 0.95% phenoxyethanol. The 0.4% xanthommatin contains 0.4% ammonium xanthommatin (Xa), 90% water, 3.5% glycerin, 0.6% carbomer, 0.4% sodium hydroxide, and 0.95% phenoxyethanol.

Test articles were patched under occlusive conditions using Finn chambers or equivalent occlusive patches. A total of nine inductions patches worn for 47 hours or 71 hours (patching occurred Mondays, Wednesdays, and Fridays) for three weeks by 60 subjects. Subjects had a rest period of 14 days. Challenge patches were applied for 48 hours and readings were made 1 hour and 48 hours post-removal.

Both formulations tested elicited no visible erythematous reactions during the Induction phase of the study. In addition, there were no questionable reactions observed during the Challenge Phase (Days 38 and 40) by any of the subjects to either test article. These results support the assessment that under the conditions of the study, the test articles have demonstrated a low potential for irritation and sensitization. Thus, the test articles can be considered safe for use under the conditions of the study, and claims such as “Dermatologically Tested”, “Clinically Tested”, “Kind to Skin” and “Safe for Skin” are substantiated.

1. An ingredient blend for use in an anti-aging product comprising an unsaturated solution of a phenoxazone and/or phenoxazine compound, or a salt thereof, wherein, upon combination with one or more further cosmetic compositions or topical dermatologic drug compositions, the ingredient blend provides an effect on one or more sign of skin-aging.

2. The ingredient blend of claim 1, wherein the phenoxazone and/or phenoxazine compound, or a salt thereof, is encapsulated in a liposome.

3. The ingredient blend of claim 2, wherein the liposome is embedded within the at least one cross-linkable polymer.

4. The ingredient blend of claim 2 or 3, wherein the liposome comprises a phospholipid and a polyol.

5. The ingredient blend of claim 4, wherein the phospholipid is selected from natural phospholipids and synthetic phospholipids.

6. The ingredient blend of claim 4, wherein the phospholipid comprises one or more of egg yolk lecithin, soybean lecithin, sphingomyelin, hydrogenated or non-hydrogenated phosphatidylcholine, phosphatidylethanolamine, phosphatidylserine, phosphatidylglycerol, phosphatidylinositol, dimyristoylphosphatidylcholine (DMPC), distearoylphosphatidylcholine, palmitoyl-stearoylphosphatidylcholine, dipalmitoyl phosphatidylcholine, and hydrogenated lecithin.

7. The ingredient blend of claim 4, wherein the phospholipid comprises lecithin.

8. The ingredient blend of claim 7, wherein the lecithin is a naturally derived unsaturated or saturated lecithin extracted from soybean or egg yolk.

9. The ingredient blend of any one of claims 4-8, wherein the polyol comprises one or more of propanediol, propylene glycol, dipropylene glycol, 1,3-butylene glycol, glycerin, methylpropanediol, isoprene glycol, pentylene glycol, erythritol, xylitol and sorbitol.

10. The ingredient blend of claim 9, wherein the polyol comprises propanediol.

11. The ingredient blend of any one of claims 2-10, wherein the liposomes are less than 1000 nm in diameter.

12. The ingredient blend of any one of claims 2-10, wherein the liposomes have a mean diameter of 250 nm.

13. The ingredient blend of any one of claims 2-10, wherein the liposomes are from about 200 to about 400 nm in diameter.

14. The ingredient blend of any one of claims 2-13, wherein the liposomes have a pH of about 5 to about 8.

15. The ingredient blend of any one of claims 1-14, wherein the phenoxazone and/or phenoxazine compound, or a salt thereof, is selected from xanthommatin, decarboxylated xanthommatin, uncyclized xanthommatin, ommatin D, dihydroxy-xanthommatin, rhodommatin, a derivative of any of the foregoing, a precursor of any of the foregoing, and salts of any of the foregoing.

16. The ingredient blend of any one of claims 1-15, wherein the unsaturated solution of a phenoxazone and/or phenoxazine compound, or a salt thereof, comprises unaggregated molecules.

17. The ingredient blend of any one of claims 1-16, wherein the phenoxazone and/or phenoxazine compound, or a salt thereof, is xanthommatin, or a salt thereof.

18. The ingredient blend of claim 17, wherein the phenoxazone and/or phenoxazine compound, or a salt thereof, is ammonium xanthommatin.

19. The ingredient blend of any one of claims 1-18, wherein the ingredient blend is water-based.

20. The ingredient blend of any one of claims 1-19, wherein the phenoxazone and/or phenoxazine compound, or a salt thereof, comprises 0.01-1 wt % of the ingredient blend.

21. The ingredient blend of any one of claims 1-19, wherein the phenoxazone and/or phenoxazine compound, or a salt thereof, compound comprises 0.4 wt % of the ingredient blend.

22. An ingredient blend for use in an anti-aging product comprising a water-based solution of xanthommatin, or a salt thereof, wherein, upon combination with one or more further cosmetic composition or topical dermatologic drug composition, the ingredient blend provides an effect on one or more sign of skin-aging, and wherein the xanthommatin, or a salt thereof, comprises up to 1 wt % of the ingredient blend.

23. The ingredient blend of claim 22, wherein the phenoxazone and/or phenoxazine compound, or a salt thereof, is encapsulated in a liposome.

24. The ingredient blend of claim 23, wherein the liposome is embedded within the at least one cross-linkable polymer.

25. The ingredient blend of claim 23 or 24, wherein the liposome comprises a phospholipid and a polyol.

26. The ingredient blend of claim 25, wherein the phospholipid is selected from natural phospholipids and synthetic phospholipids.

27. The ingredient blend of claim 25, wherein the phospholipid comprises one or more of egg yolk lecithin, soybean lecithin, sphingomyelin, hydrogenated or non-hydrogenated phosphatidylcholine, phosphatidylethanolamine, phosphatidylserine, phosphatidylglycerol, phosphatidylinositol, dimyristoylphosphatidylcholine (DMPC), distearoylphosphatidylcholine, palmitoyl-stearoylphosphatidylcholine, dipalmitoyl phosphatidylcholine, and hydrogenated lecithin.

28. The ingredient blend of claim 25, wherein the phospholipid comprises lecithin.

29. The ingredient blend of claim 28, wherein the lecithin is a naturally derived unsaturated or saturated lecithin extracted from soybean or egg yolk.

30. The ingredient blend of any one of claims 25-29, wherein the polyol comprises one or more of propanediol, propylene glycol, dipropylene glycol, 1,3-butylene glycol, glycerin, methylpropanediol, isoprene glycol, pentylene glycol, erythritol, xylitol and sorbitol.

31. The ingredient blend of claim 30, wherein the polyol comprises propanediol.

32. The ingredient blend of any one of claims 23-31, wherein the liposomes are less than 1000 nm in diameter.

33. The ingredient blend of any one of claims 23-31, wherein the liposomes have a mean diameter of 250 nm.

34. The ingredient blend of any one of claims 23-31, wherein the liposomes are from about 200 to about 400 nm in diameter.

35. The ingredient blend of any one of claims 23-34, wherein the liposomes have a pH of about 5 to about 8.

36. The ingredient blend of any one of claims 22-36, wherein the xanthommatin, or a salt thereof, comprises 0.01-1 wt % of the ingredient blend.

37. The ingredient blend of any one of claims 22-36, wherein the xanthommatin, or a salt thereof, comprises 0.4 wt % of the ingredient blend.

38. The ingredient blend of any one of claims 22-37, wherein the xanthommatin, or a salt thereof, is ammonium xanthommatin.

39. The ingredient blend of any one of claims 1-38, wherein the effect on one or more sign of skin-aging comprises the reduction, elimination, or slowing of the appearance of wrinkles.

40. The ingredient blend of any one of claims 1-38, wherein the effect on one or more sign of skin-aging comprises repairing or restoring skin elasticity or firmness.

41. The ingredient blend of any one of claims 1-38, wherein the effect on one or more sign of skin-aging comprises reduction, prevention, or slowing the appearance of hyperpigmentation.

42. The ingredient blend of any one of claims 1-38, wherein the effect on one or more sign of skin-aging comprises the reduction, slowing or elimination of dysplasias.

43. The ingredient blend of any one of claims 1-42, wherein the ingredient blend further comprises a rheology modifier.

44. The ingredient blend of claim 43, wherein the rheology modifier comprises 0.1-1 wt % of the ingredient blend.

45. The ingredient blend of claim 43, wherein the rheology modifier comprises 0.6 wt % of the ingredient blend.

46. The ingredient blend of any one of claims 43-45, wherein the rheology modifier is carbomer.

47. The ingredient blend of any one of claims 1-46, wherein the ingredient blend further comprises a humectant.

48. The ingredient blend of claim 47, wherein the humectant comprises 0.5-5 wt % of the ingredient blend.

49. The ingredient blend of claim 47, wherein the humectant comprises 3.5 wt % of the ingredient blend.

50. The ingredient blend of any one of claims 47-49, wherein the humectant is glycerin.

51. The ingredient blend of any one of claims 1-50, wherein the ingredient blend further comprises a preservative.

52. The ingredient blend of claim 51, wherein the preservative comprises 0.1-1 wt % of the ingredient blend.

53. The ingredient blend of claim 51, wherein the preservative comprises 0.95 wt % of the ingredient blend.

54. The ingredient blend of any one of claims 51-53, wherein the preservative is phenoxyethanol.

55. The ingredient blend of any one of claims 1-54, wherein the ingredient blend further comprises a pH adjuster.

56. The ingredient blend of claim 55, wherein the pH adjuster comprises 0.1-1 wt % of the ingredient blend.

57. The ingredient blend of claim 55, wherein the pH adjuster comprises 0.4 wt % of the ingredient blend.

58. The ingredient blend of any one of claims 55-57, wherein the pH adjuster is sodium hydroxide.

59. The ingredient blend of any one of claims 1-58, wherein the ingredient blend is suitable for topical administration.

60. The ingredient blend of claim 59, wherein the ingredient blend is in the form of a gel or liquid.

61. The ingredient blend of any one of claims 1-60, wherein the one or more further cosmetic compositions comprises one or more moisturizing agent, surfactant, UV absorbing agent, fragrance, anti-oxidant, body pigment, color pigments, or any other suitable general cosmetic composition.

62. The ingredient blend of claim 61, wherein the one or more further cosmetic composition comprises one or more UV absorbing agent.

63. The ingredient blend of claim 62, wherein the one or more UV-absorbing agent is selected from avobenzone, oxybenzone, oxybenzone cinoxate, homosalate, octisalate, octinoxate, octocrylene, trolamine salicylate, bemotrizinol, and bisoctrizole.

64. The ingredient blend of claim 62, wherein the one or more UV-absorbing agent is selected from kaolin, talc, petrolatum and metal oxides.

65. The ingredient blend of claim 61, wherein the one or more further cosmetic composition comprises one or more antioxidants.

66. The ingredient blend of claim 65, wherein the one or more antioxidants is selected from acetyl cysteine, alpha-lipoic acid, arbutin, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, caffeic acid, carotenoids, chlorogenic acid, cysteine, cysteine HCl, diamyihydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, flavone, flavanone, flavanol, gallic acid esters, glutathione, goosypol, hydroquinone, hydroxyanisole, isoflavones, isooctyl thioglycolate, ithiothreitol, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, protocatechuic acid, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thiodipropionic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tris(nonylphenyl)phosphite, Trolox, and xanthines.

67. The ingredient blend of claim 1-60, wherein the one or more further cosmetic composition comprises one or more agent selected from alpha and beta hydroxy acids, amino acids, peptides, matrix proteins, growth factors, stem cell activators, estrogens, anti-androgens, and skin lightening and brightening agents.

68. The ingredient blend of any one of claims 1-60, wherein the one or more topical dermatologic drug compositions is selected from anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including eflornithine and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, and wound healing agents.

69. A method of preparing a cosmetic or topical dermatologic composition for preventing or reducing the effects of skin aging in a subject, comprising combining the ingredient blend of any one of claims 1-68 and one or more further general cosmetic composition or topical dermatologic drug composition.

70. A method of preventing or reducing the effects of skin aging in a subject, comprising administering to the subject a cosmetic composition comprising the ingredient blend of any one of claims 1-68 combined with one or more further general cosmetic composition or topical dermatologic drug composition.

71. A method of stimulating collagen production in the skin of a subject, comprising administering to the subject a cosmetic composition comprising the ingredient blend of any one of claims 1-68 combined with one or more further general cosmetic composition or topical dermatologic drug composition.

72. A method of reducing, preventing, or slowing collagen degradation in the skin of a subject, comprising administering to the subject a cosmetic composition comprising the ingredient blend of any one of claims 1-68 combined with one or more further general cosmetic composition or topical dermatologic drug composition.

73. A method of stimulating keratin growth in the skin of a subject, comprising administering to the subject a cosmetic composition comprising the ingredient blend of any one of claims 1-68 combined with one or more further general cosmetic composition or topical dermatologic drug composition.

74. A method of stimulating collagen production and keratin growth in the skin of a subject, comprising administering to the subject a cosmetic composition comprising the ingredient blend of any one of claims 1-68 combined with one or more further general cosmetic composition or topical dermatologic drug composition.

75. A method of reducing, preventing, or slowing the appearance of hyperpigmentation in the skin of a subject, comprising administering to the subject a cosmetic composition comprising the ingredient blend of any one of claims 1-68 combined with one or more further general cosmetic or topical dermatologic drug ingredient.
